(19) United States
(12) Patent Application Publication
(10) Pub. No.: US 2010/0136670 A1
(43) Pub. Date: Jun. 3, 2010

(54) Sampling Method and Device

(76) Inventor: Robert J. Markovsky, Brentwood, NH (US)

Correspondence Address: CHARM SCIENCES, INC., 659 ANDOVER STREET, LAWRENCE, MA, 01843, US

(21) Appl. No.: 12/598,527

(22) PCT Filed: May 2, 2008
(86) PCT No.: PCT/US08/05710

§ 371 Date: Nov. 2, 2009

Related U.S. Application Data
(60) Provisional application No. 60/927,608, filed on May 4, 2007.

(51) Int. Cl. C12M 1/00; G01N 33/00; G01N 21/00

(52) U.S. Cl. 435/288.1; 422/68.1; 422/82.05

(57) ABSTRACT

A test device configured for obtaining a sample from within a tube such as the one or more channels of an endoscope. The device can include an elongate sample probe and light blocking material.

This application is based on and claims priority from U.S. Provisional Patent Application No. 60/927,608, filed on May 4, 2007.

### BACKGROUND

An endoscope is a device used to look inside a body cavity or organ. The scope is inserted through a natural opening or through a small incision in the skin. A medical procedure using any type of endoscope is called endoscopy. There are many types of endoscopes, and they are named in relation to the organs or areas they explore.

Some endoscopes are rigid structures employing a series of lenses, while others are flexible and employ optical fibers to illuminate the area of concern within the body and to convey an image back to the eyepiece for the surgeon to see. Surgical operating instruments may be passed into the body through the channels of the endoscope in order to perform surgical procedures such as electro-surgery or the manipulation, grasping or crushing of structures within the surgical area. Endoscope channels may also deliver fluids or gases into the surgical site or provide suction or facilitating the positioning of catheters or laser light pipes.

Following use of an endoscope in a medical procedure, a first cleaning process can be employed to remove biological soil from the outer surface of the endoscope as well as from the inner surfaces or lumen of each exposed channel. After the first cleaning step, the instrument can be disinfected using an appropriate disinfectant.

At various steps during the cleaning process it may be useful to test the endoscope to assess the effectiveness of the cleaning either on the outer surface or within the channels. One method of detecting the effectiveness of cleaning is through detection of residual adenosine triphosphate (ATP). The presence of residual ATP indicates that the cleaning was not completely effective.

One method for detecting ATP is through the use of reagents that react with ATP to generate a signal. Useful reagents include luciferin and luciferase. Such reagents can dephosphorylate ATP to produce ADP and light. Detection of such light indicates the presence of ATP.

An accurate determination of cleanliness relies on thorough sampling. Removing a sample from the outer surface of an instrument can be done using an absorbent tip. Sampling the inner surfaces of an endoscope, however, is more challenging.

There are a variety of available ATP detection devices and methods. Some such devices and methods are described in U.S. Pat. No. 5,827,675 issued Oct. 27, 1998; U.S. Pat. No. 5,965,453, issued Oct. 12, 1999; U.S. Pat. No. 6,180,395, issued Jan. 30, 2001; U.S. Pat. No. 6,055,050, issued Apr. 25, 2000; U.S. Pat. No. 5,917,592, issued Jun. 29, 1999; and U.S. Pat. No. 7,132,249, issued Nov. 7, 2006, and International Application No. PCT/US2007/001229, filed Jan. 16, 2007, all of which are incorporated herein in their entirety.

A commercially available apparatus that detects ATP is the POCKETSWAB-PLUS (POCKETSWAB is a registered trademark of Charm Sciences, Inc. of Lawrence, Mass.), which rapidly and efficiently detects ATP on surfaces. The POCKETSWAB detects ATP by emission of luminescence (light) from the reaction of luciferin and luciferase in the presence of ATP. The luminescence can be measured using a luminometer. The POCKETSWAB, and some similar devices, incorporate a foam-tipped, or other absorbent-type swab or wand, or other sampling mechanisms, for sample uptake from a surface to be monitored. Reagents for ATP detection can be located, prior to use, in a bottom reading chamber and/or in separate reagent chambers or compartments or can be located at the opposite end of the swab or elsewhere in the device and allowed to flow into the reading chamber during test operation. Some of those devices and methods can be modified, as described herein, for use in sampling endoscope channels or similar elongated structures. The POCKETSWAB style device, utilizing dark or colored plastic material to block external light penetration, combined with a luminometer with an opening that seals against the outer peripheral surface of the POCKETSWAB is particularly suited to this application. By using such a device a luminometer cap, which would be cumbersome or impossible to use with the elongated probe, is not required.

### BRIEF DESCRIPTION OF DRAWINGS

FIG. 1 is a transparent perspective view showing the pre-use position of test unit 1 with probe 2, probe pipe 11 and absorbent tip 4 extending through cover 3 and outside test unit 1.

FIG. 2 is a partially exploded, partially transparent cut away view of cover 3 with attached probe 2 and absorbent tip 4 in retracted position and removed from test unit body 12 (not shown).

FIG. 3 is a partially exploded, partially transparent view of cover 3 with attached probe 2 and absorbent tip 4 in partially extended position and removed from test unit body 12 (not shown).

FIG. 4 is a cross-sectional view of cover 3 with attached probe 2 and absorbent tip 4 in retracted position and removed from test unit body 12 (not shown).

FIG. 5 is an enlarged cross-sectional view of cover 3 with probe 2 passing through cover 3 and probe pipe 11 seated in cover 3.

FIG. 6 is an enlarged cross-sectional view of probe 2 with absorbent tip 4 and probe pipe 11.

FIG. 7 is a transparent perspective view showing a pre-use position of test unit 1 with probe 2 extending through cap and outside test unit 1 and a view of luminometer 20.

FIG. 8 is a partially transparent, perspective view of cover 3 with probe 2 covered by absorbent tip 4 being removed from test unit body 12 for use.

FIG. 9 is a perspective view of test unit cover 3 with probe 2 and absorbent tip 4 in retracted, pre-use position prior to being inserted into endoscope channel.

FIG. 10 shows absorbent tip 4 entering endoscope channel.

FIG. 11 shows the forward movement of absorbent tip 4 moving through endoscope channel.

FIG. 12 shows the backward movement of absorbent tip 4 out of endoscope channel.

FIG. 13 shows cover 3 with probe 2 and absorbent tip 4, after swabbing endoscope channel, being reinserted into test unit body 12.

FIG. 14 is a transparent view of the threadable, longitudinal movementof cover 3 over test unit body 12 and the resulting longitudinal movement of absorbent tip 4.

FIG. 15 shows test unit 1 inserted into luminometer 20 for reading.

FIG. 16 is a partially exploded, partially transparent cut away view of cover 3 with absorbent tip 4 fully covered by extended probe pipe 25. Absorbent tip 4 is in retracted position and removed from test unit body 12 (not shown).

FIG. 17 is a partially exploded, partially transparent view of cover 3 with attached probe 2 and absorbent tip 4 in partially extended position outside probe pipe extension 25 and removed from test unit body 12 (not shown).

FIG. 18 is an enlarged partial cross-sectional view of probe pipe extension 25 and absorbent tip 4 extended from out of the extension 25.

FIG. 19 is an enlarged cross-section showing probe 2 and absorbent tip 4 passing through probe pipe 11 and with absorbent tip 4 fully covered by probe pipe extension 25.

FIG. 20 is an enlarged cross-section of absorbent tip 4 puncturing first seal 26 on reading chamber 8.

FIG. 21 is an enlarged cross-section of absorbent tip 4 puncturing reagent chamber 27 seal 28 within reading chamber 8.

FIG. 22 is an enlarged cross-section showing absorbent tip 4 puncturing second reagent chamber seal 29 to release fluid or other reagents within reagent chamber 27 into bottom of reading chamber 8. Reagents 30 are shown in the bottom of reading chamber 8.

FIG. 23 is an enlarged cross-section showing absorbent tip 4 contacting reagents 30 in bottom of reading chamber 8.

### SUMMARY

Aspects include a device for detecting contamination on an internal wall of a tube, for example a cylindrical tube, such as an endoscope channel (lumen). The device can include an elongate test unit body having a solid peripheral surface. The inside of the body can be hollow, or partially hollow, to define an inner space. A removable cover can include an opening through which a probe can pass from the inside of the test unit through the cover and out of the test unit. At one end of the test unit can be a reading chamber, such as a transparent reading chamber through which light can pass and be detected by a luminometer. The reading chamber can be contiguous with the body of the test unit. Included with the test unit is an elongate probe having a first end and a second end. The first probe end can be located outside the body and the second end located, prior to use, within the inner space. The second end of the probe can include a sample collection means such as an absorbent tip. The first end of the probe can extend outside the cover opening. The cover opening can provide an interface between the inside and the outside of the test unit body. Reagents for detection of a biomolecules such as adenosine triphosphate (ATP) can be located in various areas of the device including the reading chamber and one or more optional reagent chambers. Reagents, such as luciferin and luciferase, can also be located in other areas of the device to be released after sample collection. In operation, the cover is removed from the body. When the cover is removed the probe can move, slidably, backward and forward through the cover opening and, thereby, be extended to move through, for example, a cylindrical tube such as the channel or lumen of an endoscope. After sample collection, the probe and sample collection end can be moved forward allowing the collection end, such as the absorbent tip, to move through the seals and allowing sample to contact reagents.

In various aspects the absorbent tip is used to puncture the puncturable (frangible) seals such as those enclosing a reagent chamber that is configured to retain reagents, such as liquid or solid (powder/tablet) reagents.

Various aspects also include a probe pipe. The probe pipe can provide physical support to the sample probe so that it can obtain a sample and puncture the various frangible seals of the reading chamber and/or reagent chamber. The probe pipe can have a first support end opening and a second support end opening through which the probe passes. The probe pipe can be located within the test unit body and can have a solid peripheral wall and a hollow inner space. The probe can be located within the hollow inner space. The first support end can form a continuous opening with the cap opening. The second support opening should have small enough circumference to prevent backward movement of the absorbent tip into the probe pipe.

The reading chamber of the device can include light blocking material to prevent ambient light from interfering with test results. In contrast to the light blocking provided by using dark or colored plastic, the reading chamber light blocking material is not reliant on the color of the plastic. The reading chamber light blocking material reduces light contact with test materials, for example UV radiation contact with the materials, by including a UV block material within the raw material used to make the reading chamber. Although such UV block may not completely block all UV radiation from contacting the test components, UV block can substantially reduce the amount of UV radiation contacting the test components. For example, plastic reading chambers, such as reading chambers formed from olefin based fibers such as polypropylene and polyethylene, can include UV block material incorporated into the vial plastic material.

Various aspects include a threaded device to assist the longitudinal movement of the cap and the puncturing of the various frangible seals.

### DETAILED DESCRIPTION

Some embodiments are in the format of a modified POCKETSWAB—a POCKETSWAB with an elongated absorbent tip and an elongated sample probe with three basic positions of the swab: prior to use—retracted; swabbing—partially to fully extended; and detecting—partially extended. Prior to use, the elongated probe is in the retracted position. In the retracted position the maximum length is outside of the POCKETSWAB. The probe extends through an opening in the POCKETSWAB cover. The opening can be configured to allow the probe to slide from one position to another such as from the retracted position to the fully extended position with the absorbent tip at the internal end of an endoscope channel.

To use, the absorbent-tip end is pushed into one end of an endoscope channel. As it is pushed through the channel, sample is collected onto the absorbent material. The elongated probe allows the absorbent tip to be pushed through one end of the channel and out through the other end. When the end of the channel is contacted the shaft is retracted so that the shaft returns to the pre-use position with the maximum length outside the POCKETSWAB.

When moved back from the fully extended to the retracted, pre-use position the cover can be reconnected to the body. The absorbent-tip can next be used to puncture frangible seals covering one or more reagent chambers, in the form of one or more cylinders containing reagents and sealed on both ends with a probe puncturable membrane, releasing and activating the necessary reagents to detect the presence of ATP.

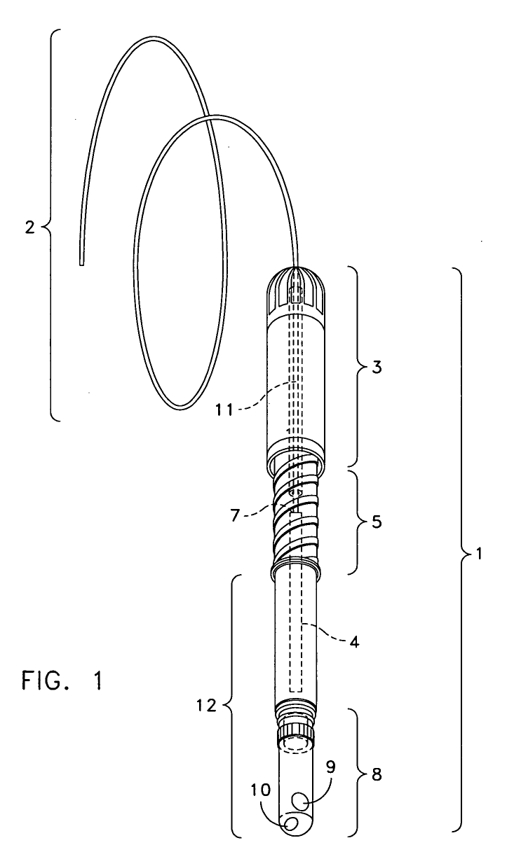

FIG. 1 shows an embodiment of test unit 1 in the pre-use (full retracted) position. Probe 2 extends from within absorbent tip 4 through probe pipe 11 and out through cover 3. In pre-use position proximal end 7 of absorbent tip 4 is seated against exposed end 6 of probe pipe 11. Probe pipe 11 is secured within cover 3. Threads 5 of this embodiment allow for the controlled longitudinal movement of cover 3. Reagent tablets 9, 10 are shown in the bottom reading chamber 8. Reagents can be in a variety of forms including tablets (as shown), liquid and powder.

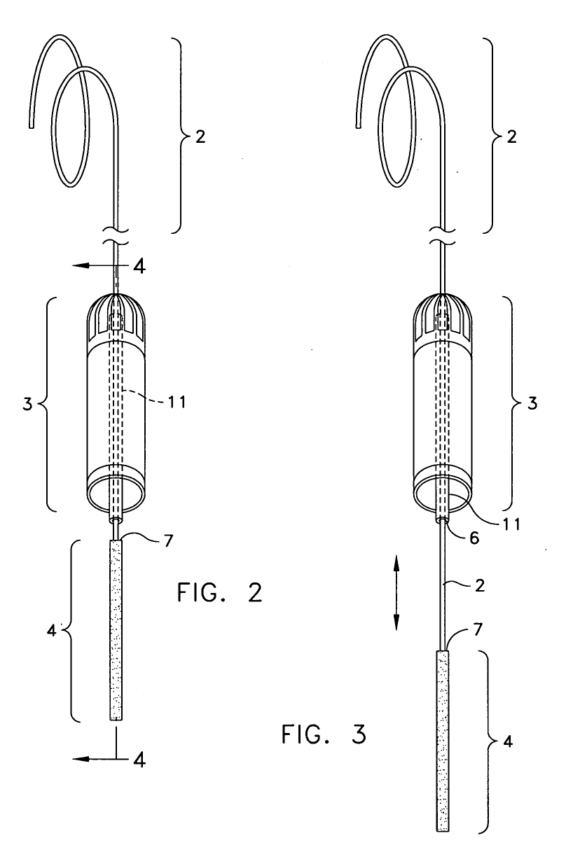

FIG. 2 shows cover portion 3 and absorbent tip 4 separated from the test unit body 12 (not shown) and in the fully retracted position. FIG. 3 shows the movement of absorbent tip 4 away from probe pipe 11 causing an extension of probe 2 section between exposed end 6 of probe pipe 11 and absorbent tip 4 as probe 2 is slid from outside cover 3 through probe pipe 11 causing extension of probe 2 from probe pipe 11.

The absorbent tip 4 can include an elongate tube of absorbent material that can be wrapped around and/or attached, such as adhesively attached, to the probe 2. A variety of materials can be used including foam such as polyurethane foam. Other useful materials include any type of porous material including rayon, Dacron, cotton or a combination thereof. The probe can be pre-moistened with any one or more of a variety of liquids, depending on the need or application, or provided dry. If provided pre-moistened, the pre-moistening liquid can include, for example, buffer, sterile water, glycerin, diluents, wetting solutions, or other material desired to be mixed with the sample or useful for absorbing, neutralizing, stabilizing or maintaining a sample.

To provide supporting structure a probe pipe can be included. In an embodiment, a probe pipe can be seated within cover. The probe and probe pipe can be composed of a variety of materials, including plastic such as polypropylene based plastic.

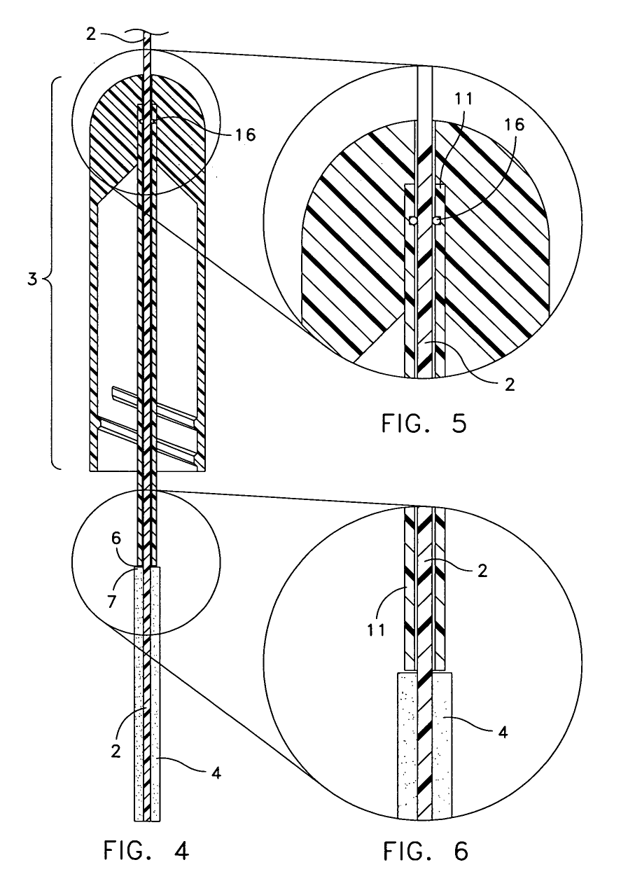

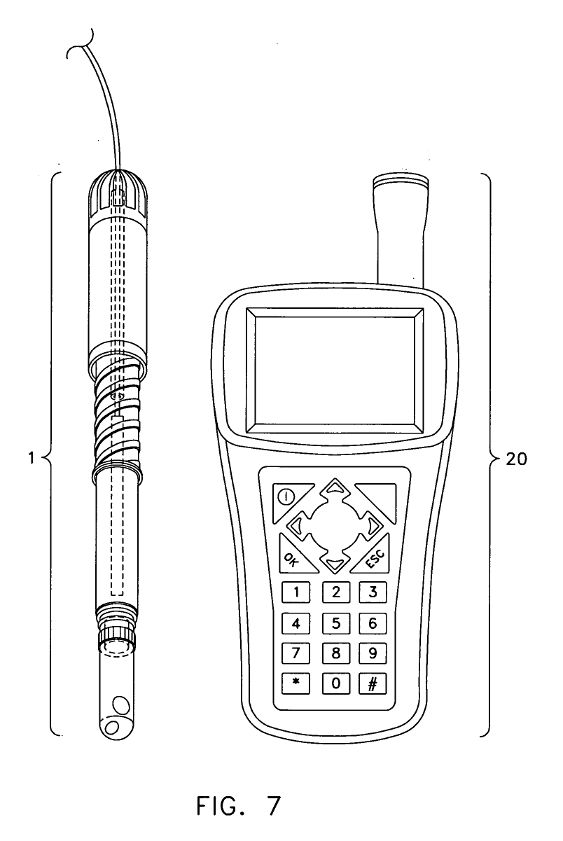

The cross-sectional views of FIG. 4, FIG. 5 and FIG. 6 show an embodiment with probe pipe 11 seated within cover 3. The figures show the seating of probe pipe 11 within cover 3 thereby allowing probe 2 to slidably extend through cover 3, being supported within cover 3 by probe pipe 11. Open end 6 of probe pipe 11 provides a barrier against which proximal end 7 of absorbent tip 4 can abut to prevent probe 2 from retracting completely out of test unit 1. Optional o-ring 16 prevents light leakage into reading chamber. Optional o-ring can be useful alone or in combination with, for example, a probe such as a probe composed of black plastic. Black probe may be sufficient, without o-ring, to block light from interfering with testing.

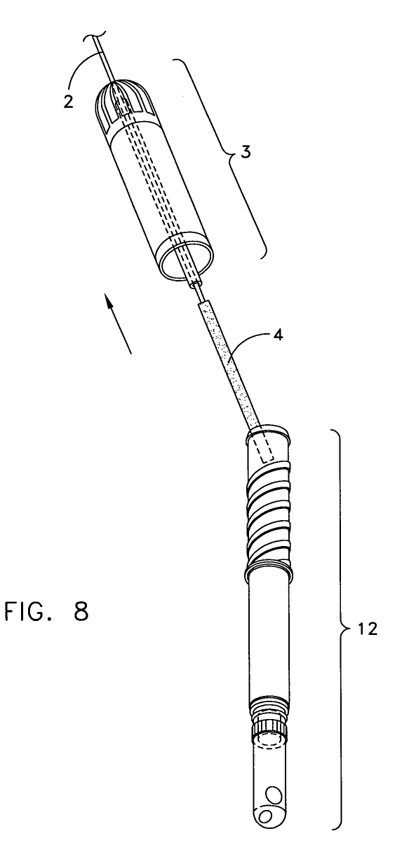

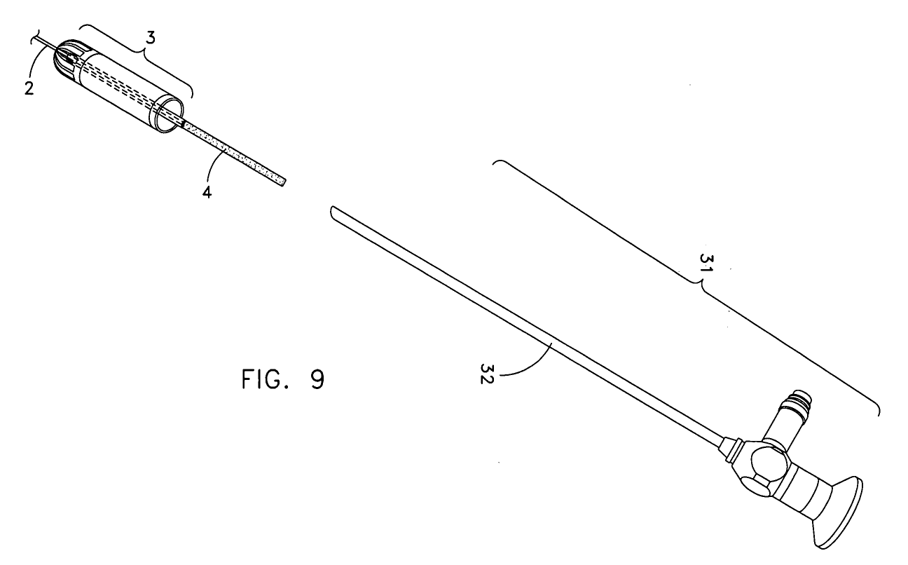

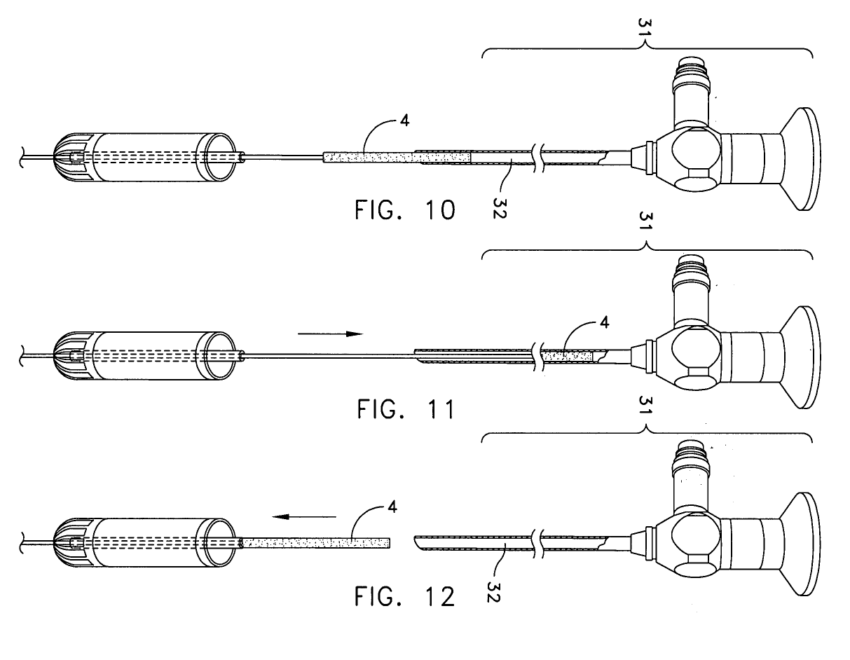

FIG. 8 shows cover 3 with absorbent tip 4 being removed from test unit body 12 prior to use such as for obtaining a sample from within an endoscope. In operation, as shown in FIGS. 9, 10, 11 and 12, absorbent tip 4 is inserted into endoscope 31 by extending probe 2 through the length of endoscope and then retracting probe 2 back to the retracted, pre-use position. If the endoscope channel 32 is not clean residual material will be retained on absorbent tip 4.

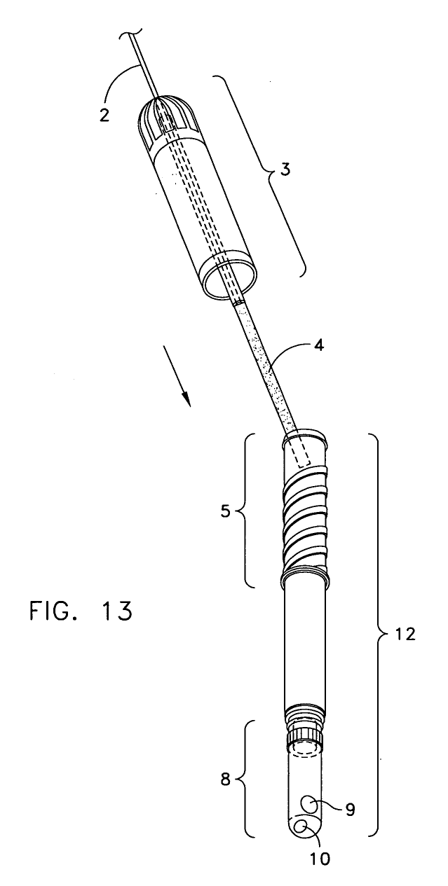

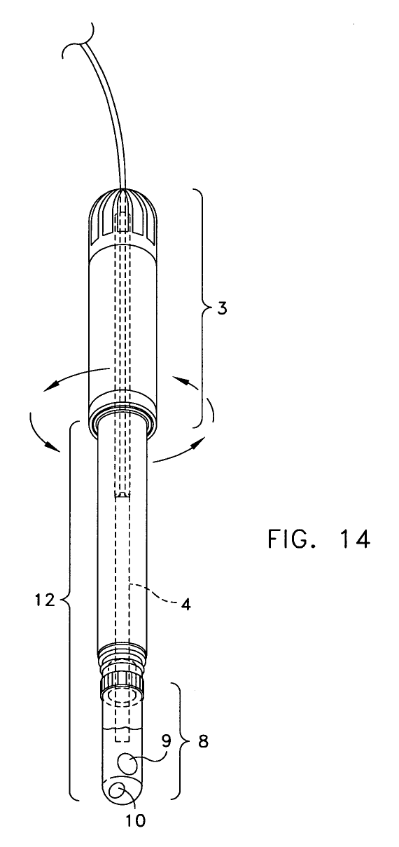

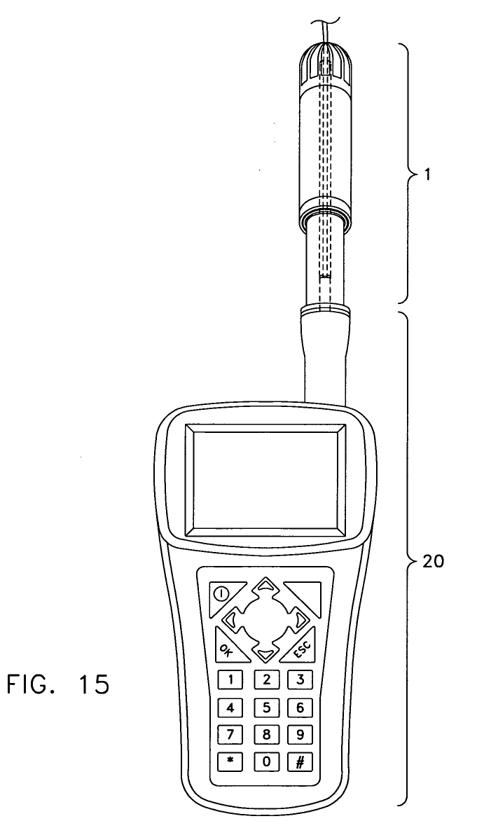

FIGS. 13 and 14 show that after retraction cover 3 can be replaced onto test unit body 12. In embodiments including threads 5, swab cover 3 can be screwed downwardly so that absorbent tip 4 is used to release liquid from within a reagent chamber. Compartments can be sealed with frangible seals so that rupturing of the frangible seals releases reagents into reading chamber. Reagents can be located within reagent chambers and/or the reading chamber 8 to be combined with the sample from absorbent tip. FIGS. 13 and 14 show reagents 9, 10, in optional tablet form, within reading chamber 8.

If the reagents are for detection of ATP, such as luciferin/luciferase, and the endoscope channel has not been sufficiently cleaned, ATP from the sample will combine with the reagents to generate light. FIG. 15 shows the test unit inserted into a reader such as a luminometer 20 so that the reading chamber 8 is in position relative to the light detection mechanism of the luminometer 20 so that the luminometer 20 can detect light output. A variety of luminescence detectors (luminometers), including photomultiplier tube and/or photodiode based detectors, can be used to read the luminescent output. The luminescence reader may, for example, be in the format of the LUMINATOR-K, LUMINATOR-T, FIREFLY, LUM-96, LUMGIENE and NOVALUM readers (LUMINATOR-K, LUMINATOR-T, FIREFLY, LUM-96, LUMGIENE AND NOVALUM are trademarks of Charm Sciences, Inc.; Lawrence, Mass.) The luminescence reader may also be in the format of anyluminescence reading device such as a photodiode, or a photomultiplier based luminometer.

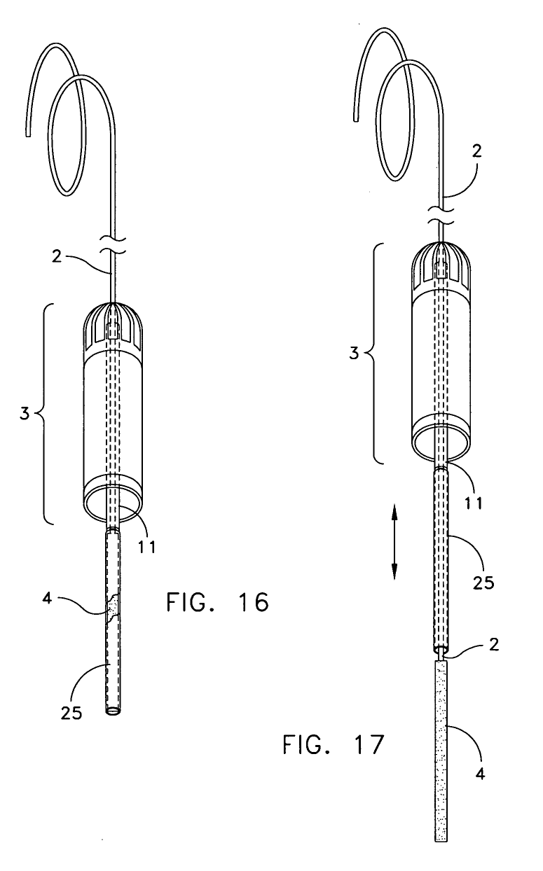

FIGS. 16 through 19 show an embodiment including probe pipe extension. Generally, probe 2 is sufficiently rigid to puncture the various foils seals that may be used to separate and store reagents within test unit 1 such as within reagent chambers and/or the reading chamber 8. In some cases, however, it may be useful to provide a probe pipe extension 25. Extension covers absorbent tip 4 and provides additional strength for puncturing one or more frangible seals. In some embodiments an extension abuts probe pipe 11 and has a slightly larger diameter than probe pipe 11. In that way absorbent tip 4 can slide out of and into extension and not slide through probe pipe 11.

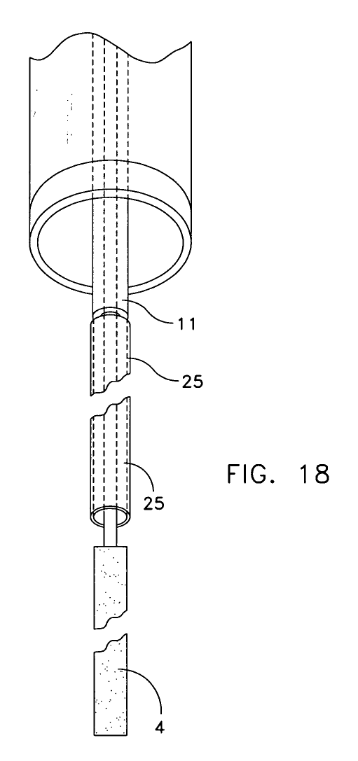

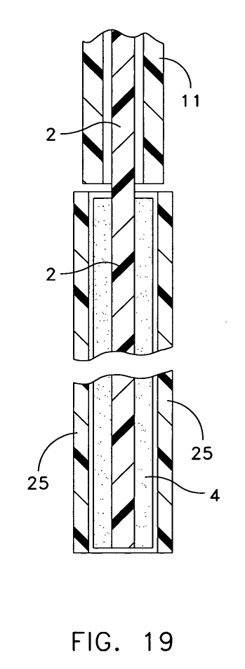

FIG. 16 shows extension covering absorbent tip 4 when probe 2 is in the retracted position. FIG. 17 shows absorbent tip 4 extending out from extension. FIG. 18 and FIG. 19 show extension 25 with a slightly larger internal diameter than probe pipe 11. FIG. 19 shows that in relation to probe pipe 11 and also shows probe pipe 11 with a slightly smaller internal diameter than absorbent tip 4 so that absorbent tip 4 cannot retract through probe pipe 11.

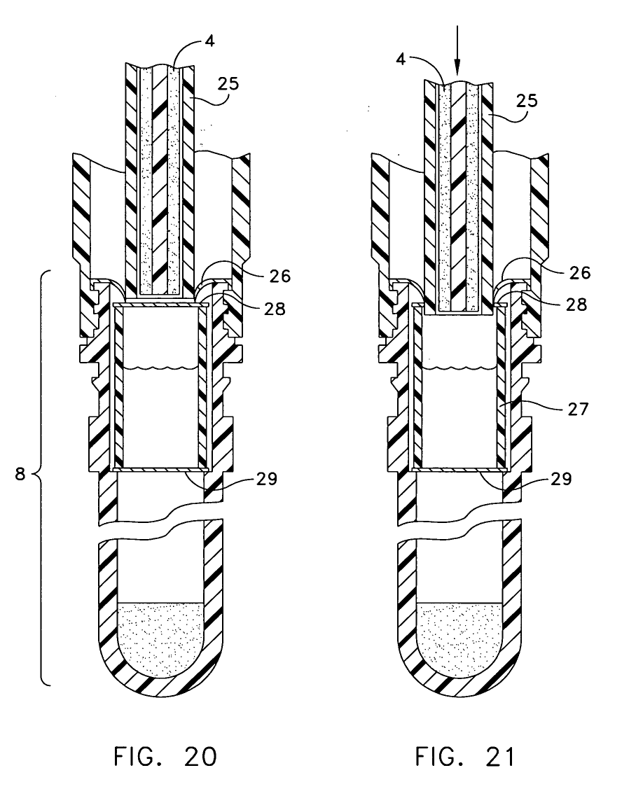

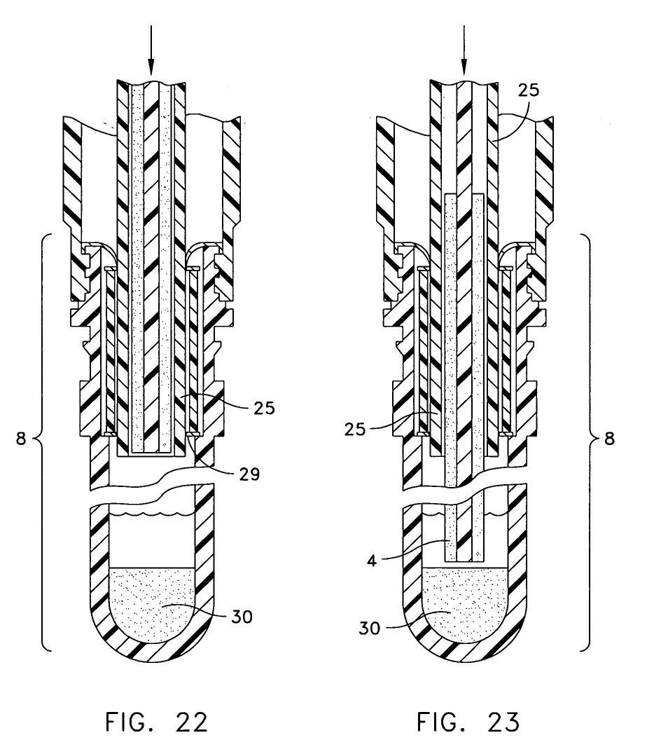

FIGS. 20 through 23 show the absorbent tip 4 being used to puncture three separate seals: reading chamber seal 26, top reagent chamber seal 28 and bottom reagent chamber seal 29. Puncturing of the seals, and contact of absorbent tip 4 with the reagents 30, allows reagents 30 to combine with sample for detection as shown.

Reagent chambers can be composed of a variety of materials such as organic polymeric materials including polypropylene, polyethylene, polybutyrate, polyvinylchloride and polyurethane. Reagent chamber and reagent chamber materials can be irradiated or otherwise treated to reduce or eliminate possible contamination. Reagent chambers can be a variety of sizes to hold a variety of quantities or volumes. Reagents within optional reagent chambers can include a variety of materials depending on the test to be run. The materials within the reagent chambers may be in the form of a solid, liquid, powder, emulsion, suspension, tablet or any combination thereof. One or more reagent chambers can be provided either within the top portion of the reading chamber or above the reading chamber within other portions of the test unit. Reagent chambers can be sealed on both sides with frangible, puncturable seals. The seals can be a variety or combination of organic polymeric materials such as silicone, rubber, polyurethane, polyvinylchloride or inorganic material such as wax or foil material. Use of optional reagent chambers allows additional reagents or reagent combinations to be provided with the test unit separate from the reagents within the reading chamber. During test operation the reagents within the reagent chamber are contacted by puncturing the seal for example with the absorbent tip 4 or the extension 25.

Reagent and/or reading chambers can include a material that reduces the amount of ultraviolet (“UV”) radiation that can penetrate the chamber, such as a UV blocking/filtering material (“UV block”) that can be mixed into the test vial raw material. The reading and/or reagent chamber raw material can include a combination of plastic materials, including, for example, polypropylene and polyethylene. The UV block can be, for example, CIBA Shelf life Plus UV1100. Typical combinations include about 99.5% plastic to about 0.5% UV block for example, 0.5% Ciba SHELFPLUS UV 1100 combined with 99.5% Marlex RLC-350 (clarified polypropylene random copolymer, antistatic, controlled rheology). Other UV filter material may be usefully employed such as the variety available from CIBA. Generally, UV block material and/or light blocking covers may be useful to limit interference with any of the variety of test apparatus and methods in which luminescent signals provide results. These filtering and/or blocking techniques may be particularly useful when operating at the limits of sensitivity and selectivity.

1. A device for detecting contamination on an internal wall of a tube, the device comprising:
a) an elongate test unit body having a solid peripheral surface, a hollow, or partially hollow, inner space and a removable cover, the cover having an opening;
b) a reading chamber at one end of the body and contiguous with the body;
c) an elongate probe, the probe having a first end and a second end, the first end located outside the body and the second end located, prior to use, within the inner space, the probe including an absorbent tip at the second end, the cover opening providing the probe interface between the inside and the outside of the body; and
d) at least one reagent for detecting a biomolecule,
wherein when the cover is removed from the body the probe can slidably move backward and forward through the cover opening, the forward movement allowing the absorbent tip to move through the tube and the backward movement allowing the absorbent tip to be removed from the tube.

2. The device of claim 1 wherein the biomolecule to be detected comprises adenosine triphosphate.

3. The device of claim 1 further comprising a plurality of reagents for detecting a biomolecule.

4. The device of claim 3 wherein the reagents comprise luciferin and luciferase.

5. The device of claim 4 wherein the luciferin and luciferase are within the reading chamber.

6. The device of claim 1 wherein the reagents are enclosed within the reading chamber by a puncturable seal.

7. The device of claim 1 further comprising a reagent chamber, the reagent chamber configured to retain reagents, the chamber formed using puncturable seals.

8. The device of claim 7 wherein the reagents stored within the reagent chamber comprise a liquid.

9. The device of claim 7 wherein the reagents stored within the reagent chamber comprise a solid.

10. The device of claim 1 further comprising a probe pipe, the probe pipe providing probe support and having a first support end opening and a second support end opening, the probe support located within the body and having a solid peripheral wall and a hollow inner space containing the probe, the first support end opening forming a continuous opening with the cap opening and the second support opening, the second support opening configured to limit backward movement of the probe.

11. The device of claim 1 wherein the test unit body comprises a dark colored plastic.

12. The device of claim 1 wherein the probe comprises a dark colored plastic.

13. The device of any one of claims 11 and 12 wherein the dark colored plastic material comprises black colored plastic.

14. The device of claim 1 wherein the tube comprises an endoscope.

15. The device of claim 1 wherein the cap and test unit body are arranged and configured for longitudinal movement of the cap.

16. The device of claim 1 in combination with a luminometer, the luminometer configured with an opening that seals against the solid peripheral surface of the test unit body.

17. The device of claim 1 wherein the reading chamber comprises a UV blocking material.

18. The device of claim 1 wherein the reading chamber comprises a combination of a plastic material and a UV blocking material.

19. The device of claim 18 wherein the plastic material is combined with the UV blocking material at a ratio of about 99.5% plastic to about 0.5% UV block.

20. The device of claim 18 wherein the plastic material comprises polypropylene.

21. The device of claim 17 wherein the UV blocking material comprises CIBA Shelflife Plus.
